(19) United States
(12) Patent Application Publication
(10) Pub. No.: US 2019/0217060 A1
(43) Pub. Date: Jul. 18, 2019

(54) HAND CONTROL ASSEMBLY AND METHOD

(71) Applicant: Cryterion Medical, Inc., Carlsbad, CA (US)

(72) Inventor: Chadi Harmouche, Saint-Laurent (CA)

(21) Appl. No.: 16/240,400

(22) Filed: Jan. 4, 2019

Related U.S. Application Data
(60) Provisional application No. 62/618,481, filed on Jan. 17, 2018.

(51) Int. Cl. A61M 25/01; A61B 18/14; A61M 25/10

(52) U.S. Cl. CPC A61M 25/0136; A61B 18/1492; A61B 2018/0022; A61M 25/10184; A61M 25/104; A61M 25/0116

(57) ABSTRACT

A hand control assembly for an intravascular catheter system includes a controller and a plurality of spaced apart hand-actuated members. The intravascular catheter system includes a control console that is configured to be positioned on a support surface. Each hand-actuated member is configured to be manually actuated and is positioned away from the control console and support surface. The hand control assembly controls varying stages of an ablation procedure, including an inflation stage, ablation stage, thawing stage and/or time to isolation. Each hand-actuated member sends at least one (i) initiation signal to the controller to initiate at least one of the inflation stage, ablation stage and/or a calculation of time to isolation, and/or (ii) termination signal to the controller to terminate at least one of the inflation stage, ablation stage and/or thawing stage.

### CROSS-REFERENCE TO RELATED APPLICATION

This application claims priority to U.S. Provisional Application Ser. No. 62/618,481 filed on Jan. 17, 2018 and entitled “HAND CONTROL ASSEMBLY AND METHOD”. As far as permitted, the contents of U.S. Provisional Application Ser. No. 62/618,481 are incorporated in their entirety herein by reference.

### TECHNICAL FIELD

The present disclosure relates to medical devices and methods for treating cardiac arrhythmias. More specifically, the disclosure relates to devices and methods for cardiac cryoablation.

### BACKGROUND

Cardiac arrhythmias involve an abnormality in the electrical conduction of the heart and are a leading cause of stroke, heart disease, and sudden cardiac death. Treatment options for patients with arrhythmias include medications, implantable devices, and catheter ablation of cardiac tissue.

Catheter ablation involves delivering ablative energy to tissue inside the heart to block aberrant electrical activity from depolarizing heart muscle cells out of synchrony with the heart's normal conduction pattern. The procedure is performed by positioning a portion of an energy delivery catheter adjacent to diseased or targeted tissue in the heart. The energy delivery component of the system is typically at or near a most distal (farthest from the user) portion of the catheter, and often at a tip of the device. Various forms of energy are used to ablate diseased heart tissue. These can include radio frequency (RF), ultrasound and laser energy, to name a few. One form of energy that is used to ablate diseased heart tissue includes cryogenics (also referred to herein as “cryoablation”). During an ablation procedure, with the aid of a guidewire, the distal tip of the catheter is positioned adjacent to diseased or targeted tissue, at which time the cryogenic energy can be delivered to create tissue necrosis, rendering the ablated tissue incapable of conducting electrical signals.

Atrial fibrillation is one of the most common arrhythmias treated using cryoablation. In the earliest stages of the disease, paroxysmal atrial fibrillation, the treatment strategy involves isolating the pulmonary veins from the left atrial chamber, a procedure that removes unusual electrical conductivity in the pulmonary vein. Recently, the use of techniques known as “balloon cryotherapy” catheter procedures to treat atrial fibrillation have increased. In part, this stems from ease of use, shorter procedure times and improved patient outcomes. During the balloon cryotherapy procedure, a refrigerant or cryogenic fluid (such as nitrous oxide, or any other suitable fluid) is delivered under pressure to an interior of one or more inflatable balloons which are positioned adjacent to or against the targeted cardiac tissue. Using this method, the extremely frigid cryogenic fluid causes necrosis of the targeted cardiac tissue, thereby rendering the ablated tissue incapable of conducting unwanted electrical signals.

Ablation procedures generally require the use of multiple hand-held and/or hand-controlled structures or devices. For example, a handle assembly may be handled or used by a user to operate, position and/or control a catheter. Furthermore, a control console may often include various structures, components or devices, including a graphical display, which may require the user's manual control, guidance and/or input. More specifically, the control console requires the use of a hand or hands, which can lead to a relatively non-sterile environment during the ablation procedure. There is a continuing need to improve the operability of cryogenic ablation systems.

### SUMMARY

The present disclosure is directed towards a hand control assembly for an intravascular catheter system that is used during at least one stage of an ablation procedure. In certain embodiments, the intravascular catheter system can include a handle assembly and/or a control console. The control console is configured to be positioned on a support surface. The hand control assembly can include a controller and a plurality of spaced apart hand-actuated members. The plurality of hand-actuated members are each configured to be manually actuated by a user. Each hand-actuated member is positioned away from the control console and the support surface. As non-exclusive examples, at least one hand-actuated member can be positioned on the handle assembly and/or positioned away from the handle assembly. Alternatively, each hand-actuated member can be positioned away from the handle assembly, the control console and the support surface. Still alternatively, each hand-actuated member can be positioned on the handle assembly. Additionally, each hand-actuated member can send at least one initiation signal to the controller to initiate at least one stage of the ablation procedure, and/or termination signal to the controller to terminate at least one stage of the ablation procedure.

In various embodiments, the plurality of hand-actuated members can include a first hand-actuated member and a second hand-actuated member. In one embodiment, the first hand-actuated member and the second hand-actuated member can both be configured to be manually actuated by the user to send at least one initiation signal to the controller to initiate at least one stage of the ablation procedure. In another embodiment, the first hand-actuated member and the second hand-actuated member can each be configured to be manually actuated by the user to send at least one termination signal to the controller to terminate at least one stage of the ablation procedure. In still another embodiment, the first hand-actuated member can be configured to be manually actuated by the user to send at least one initiation signal to the controller to initiate at least one stage of the ablation procedure and the second hand-actuated member can be configured to be manually actuated by the user to send at least one termination signal to the controller to terminate at least one stage of the ablation procedure. Alternatively, the first hand-actuated member can be configured to be manually actuated by the user to send at least one termination signal to the controller to terminate at least one stage of the ablation procedure and the second hand-actuated member can be configured to be manually actuated by the user to send at least one initiation signal to the controller to initiate at least one stage of the ablation procedure.

In certain embodiments, at least one of the plurality of hand-actuated members can include at least one of a button or a switch.

In certain embodiments, the ablation procedure can include an inflation stage. In one embodiment, the initiation signal can initiate the inflation stage. In another embodiment, the termination signal can terminate the inflation stage.

In some embodiments, the ablation procedure can include an ablation stage. In one embodiment, the initiation signal can initiate the ablation stage. In another embodiment, the termination signal can terminate the ablation stage.

In other embodiments, the ablation procedure can include a time to isolation. In one embodiment, the initiation signal can initiate a calculation of the time to isolation.

In still other embodiments, the ablation procedure can include a thawing stage. In one embodiment, the termination signal can terminate the ablation stage and can substantially simultaneously initiate the thawing stage. Alternatively, the termination signal can terminate the thawing stage.

In some embodiments, at least one hand-actuated member can be configured to be manually actuated by the user to send a timer signal to the controller to initiate a timer. In alternative embodiments, at least one hand-actuated member can be configured to be manually actuated by the user to send at least one of a deactivation signal to deactivate the hand control assembly, and/or an activation signal to activate the hand control assembly.

In certain embodiments, the hand control assembly may further include a member support surface. At least one of the plurality of hand-actuated members is supported by the member support surface. In some embodiments, the member support surface can be moveable relative to the support surface.

In various embodiments, the controller may be positioned within the control console.

The present disclosure is also directed toward a method for controlling at least one stage of an ablation procedure. The method can include the steps of positioning each of a plurality of hand-actuated members at a location that includes on a handle assembly, away from a control console and a support surface, and/or away from the handle assembly, the control console and the support surface; and sending with each of the plurality of hand-actuated members at least one initiation signal to the controller to initiate at least one stage of the ablation procedure, and/or termination signal to the controller to terminate at least one stage of the ablation procedure.

In one embodiment, the step of sending can include sending the initiation signal to the controller to initiate an inflation stage. In another embodiment, the step of sending can include sending the initiation signal to the controller to initiate an ablation stage. In still another embodiment, the step of sending can include sending the initiation signal to the controller to initiate a calculation of a time to isolation. In yet another embodiment, the step of sending can include sending the termination signal to the controller to terminate the inflation stage. In even another embodiment, the step of sending can include sending the termination signal to the controller to terminate the ablation stage. Alternatively, the step of sending can include sending the termination signal to the controller to terminate the ablation stage and substantially simultaneously initiate a thawing stage. Still alternatively, the step of sending can include sending the termination signal to the controller to terminate the thawing stage.

In one embodiment, the method can include the step of supporting with a member support surface at least one of the plurality of hand-actuated members.

Further, the method can also include the step of sending with at least one hand-actuated member a timer signal to the controller to initiate a timer. Alternatively, the method can include the step of sending with at least one hand-actuated member a deactivation signal to the controller to deactivate the hand control assembly, and/or an activation signal to the controller to activate the hand control assembly.

Additionally, the present disclosure is also directed toward a hand control assembly for an intravascular catheter system that is used during least one stage of an ablation procedure. In certain embodiments, the intravascular catheter system can include a control console that is configured to be positioned on a support surface. The hand control assembly can include a controller and a first hand-actuated member that is configured to be manually actuated by a user following a first hand-actuated member sequence. The first hand-actuated member is positioned away from the control console and the support surface. Additionally, the first hand-actuated member sends at least a plurality of initiation signals to the controller to initiate at least one stage of the ablation procedure, and/or a plurality of termination signals to the controller to terminate at least one stage of the ablation procedure.

In one embodiment, the first hand-actuated member sequence can be predetermined by the user. In another embodiment, the first hand-actuated member sequence can be preprogrammed.

The ablation procedure includes an inflation stage, an ablation stage, a time to isolation and a thawing stage, as non-exclusive examples. In certain embodiments, the first hand-actuated member can be manually actuated a first time by the user to send a first initiation signal to the controller to initiate the inflation stage. In various embodiments, the first hand-actuated member can be manually actuated a plurality of times to send a second initiation signal to the controller to initiate the ablation stage. In some embodiments, the first hand-actuated member can be manually actuated the plurality of times to send a third initiation signal to the controller to initiate a calculation of the time to isolation. In other embodiments, the first hand-actuated member can be manually actuated the plurality of times to send a first termination signal to the controller to terminate the inflation stage. In still other embodiments, the first hand-actuated member can be manually actuated the plurality of times to send a second termination signal to the controller to terminate the ablation stage. In yet other embodiments, the first hand-actuated member can be manually actuated the plurality of times to send a third termination signal to the controller to terminate the thawing stage. Additionally, the first hand-actuated member can be manually actuated the plurality of times to send a timer signal to the controller to initiate a timer.

In some embodiments, the hand control assembly can include a second hand-actuated member that is configured to be manually actuated by the user following a second hand-actuated member sequence to send the timer signal to the controller to initiate the timer.

In other embodiments, the hand control assembly can include the second hand-actuated member that is configured to be manually actuated by the user following the second hand-actuated member sequence to send a deactivation signal to the controller to deactivate the hand control assembly, and/or an activation signal to the controller to activate the hand control assembly. For example, the second hand-actuated member can be actuated a first time to send the deactivation signal to the controller to deactivate the hand control assembly. Further, the second hand-actuated member can be actuated a plurality of times to send the activation signal to the controller to activate the hand control assembly.

In various embodiments, the hand control assembly can include the second hand-actuated member that is configured to be manually actuated by the user following the second hand-actuated member sequence to send the plurality of initiation signals to the controller to initiate at least one stage of the ablation procedure, and/or the plurality termination signals to the controller to terminate at least one stage of the ablation procedure. In one embodiment, the second hand-actuated member can be actuated a first time during the inflation stage to send the first termination signal to the controller to terminate the inflation stage. In another embodiment, the second hand-actuated member can be actuated a first time during the ablation stage to send the second termination signal to the controller to terminate the ablation stage. Alternatively, the second hand-actuated member can be actuated a first time during the ablation stage to send the second termination signal to the controller to terminate the ablation stage and substantially simultaneously initiate the thawing stage. In yet another embodiment, the second hand-actuated member can be actuated a first time during the thawing stage to send the third termination signal to the controller to terminate the thawing stage.

In certain embodiments, the intravascular catheter system can include a handle assembly. In one embodiment, the first hand-actuated member can be positioned away from the handle assembly, the control console and the support surface. In another embodiment, the first hand-actuated member can be positioned on the handle assembly.

In various embodiments, the hand control assembly may further include a member support surface. The first hand-actuated member can be supported by the member support surface. In some embodiments, the member support surface can be moveable relative to the support surface.

In other embodiments, the controller may be positioned within the control console.

The present disclosure is further directed toward a method for controlling at least one stage of an ablation procedure. The method can include the steps of positioning a first hand-actuated member at a location that includes on a handle assembly, away from a control console and a support surface, and/or away from the handle assembly, the control console and the support surface; and manually actuating the first hand-actuated member following a first hand-actuated member sequence to send at least one of a plurality of initiation signals to the controller to initiate at least one stage of the ablation procedure, and/or a plurality of termination signals to the controller to terminate at least one stage of the ablation procedure.

In various embodiments, the step of manually actuating can include actuating the first hand-actuated member a first time to send a first initiation signal to the controller to initiate an inflation stage. In some embodiments, the step of manually actuating can include actuating the first hand-actuated member a plurality of times to send a second initiation signal to the controller to initiate an ablation stage. In other embodiments, the step of manual actuating can include actuating the first hand-actuated member the plurality of times to send a third initiation signal to the controller to calculate a time to isolation. In still other embodiments, the step of manual actuating can include actuating the first hand-actuated member the plurality of times to send a first termination signal to the controller to terminate the inflation stage. In yet other embodiments, the step of manual actuating can include actuating the first hand-actuated member the plurality of times to send a second termination signal to the controller to terminate the ablation stage. In even other embodiments, the step of manual actuating can include actuating the first hand-actuated member the plurality of times to send a third termination signal to the controller to terminate a thawing stage.

In one embodiment, the method can further include the step of supporting with a member support surface the first hand-actuated member.

In some embodiments, the method can include the step of manually actuating a second hand-actuated member following a second hand-actuated member sequence to send a timer signal to the controller to initiate a timer.

In other embodiments, the method can further include the step of manually actuating the second hand-actuated member following the second hand-actuated member sequence to send a deactivation signal to the controller to deactivate the hand control assembly, and/or an activation signal to the controller to activate the hand control assembly. In one embodiment, the step of manually actuating can include actuating the second hand-actuated member a first time to send the deactivation signal to the controller to deactivate the hand control assembly. In another embodiment, the step of manually actuating can include actuating the second hand-actuated member a plurality of times to send the activation signal to the controller to activate the hand control assembly.

In certain embodiments, the method can also include the step of manually actuating the second hand-actuated member following the second hand-actuated member sequence to send the plurality of initiation signals to the controller to initiate at least one stage of the ablation procedure, and/or the plurality of termination signals to the controller to terminate at least one stage of the ablation procedure. In one embodiment, the step of manually actuating can include actuating the second hand-actuated member a first time during the inflation stage to send the first termination signal to the controller to terminate the inflation stage. In another embodiment, the step of manually actuating can include actuating the second hand-actuated member a first time during the ablation stage to send the second termination signal to the controller to terminate the ablation stage. Alternatively, the step of manually actuating can include actuating the second hand-actuated member a first time during the ablation stage to send the second termination signal to the controller to terminate the ablation stage and substantially simultaneously initiate the thawing stage. In still another embodiment, the step of manually actuating can include actuating the second hand-actuated member a first time during the thawing stage to send the third termination signal to the controller to terminate the thawing stage.

Additionally, the method can further include the step of manually actuating a third hand-actuated member following a third hand-actuated member sequence to send the timer signal to the controller to initiate the timer.

Further, in some applications, the present disclosure is directed toward a hand control assembly for an intravascular catheter system that is used to control a flow rate of a cryogenic fluid to a balloon catheter. In certain embodiments, the intravascular catheter system can include a control console that is configured to be positioned on a support surface. The hand control assembly can include a controller and a first hand-actuated member that is configured to be manually actuated by a user. The first hand-actuated member is positioned away from the control console and the support surface. Further, the first hand-actuated member sends a first depression signal to the controller to control the flow rate of the cryogenic fluid to the balloon catheter when the first hand-actuated member is depressed and held down. The first hand-actuated member sends a first release signal to the controller to maintain the flow rate of the cryogenic fluid to the balloon catheter when the first hand-actuated member is released.

In various embodiments, the first depression signal can be sent to the controller each time the first hand-actuated member is depressed and held down. Further, the first release signal can be sent to the controller each time the first hand-actuated member is released.

In some embodiments, the hand control assembly can further include a second hand-actuated member that is configured to be manually actuated by the user. The second hand-actuated member sends a second depression signal to the controller to control the flow rate of the cryogenic fluid to the balloon catheter when the second hand-actuated member is depressed and held down. The second hand-actuated member sends a second release signal to the controller to maintain the flow rate of the cryogenic fluid to the balloon catheter when the second hand-actuated member is released.

In various embodiments, the intravascular catheter system can include a handle assembly. In certain embodiments, the first hand-actuated member and/or the second hand-actuated member can be positioned away from the handle assembly, the control console and/or the support surface. In other embodiments, the first hand-actuated member and/or the second hand-actuated member can be positioned on the handle assembly.

In certain embodiments, the hand control assembly may further include a member support surface. At least one of the first hand-actuated member and the second hand-actuated member can be supported by the member support surface. In some embodiments, the member support surface can be moveable relative to the support surface.

In various embodiments, the controller can be positioned within the control console.

The present disclosure is further directed toward a method for controlling a flow rate of a cryogenic fluid to a balloon catheter. The method can include the steps of positioning a first hand-actuated member at a location that includes on a handle assembly, away from a control console and a support surface, or away from the handle assembly, the control console and the support surface; and manually actuating the first hand-actuated member to send at least one first depression signal to the controller to control the flow rate of the cryogenic fluid to the balloon catheter, and/or first release signal to the controller to maintain the flow rate of the cryogenic fluid to the balloon catheter.

In certain embodiments, the step of manually actuating can include depressing and holding down the first hand-actuated member and/or releasing the first hand-actuated member.

In some embodiments, the method can also include the step of manually actuating a second hand-actuated member to send at least one second depression signal to the controller to control the flow rate of the cryogenic fluid to the balloon catheter, and/or second release signal to the controller to maintain the flow rate of the cryogenic fluid to the balloon catheter.

In one embodiment, the method can further include the step of supporting with a member surface at least one of the first hand-actuated member and the second hand-actuated member.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a schematic side view of a patient, a user and an embodiment of an intravascular catheter system having features of the present disclosure, including one embodiment of a hand control assembly;

FIG. 2 is a schematic side view of the patient, the user and another embodiment of the intravascular catheter system, including another embodiment of the hand control assembly;

FIG. 3 is a schematic side view of the patient, the user and still another embodiment of the intravascular catheter system, including still another embodiment of the hand control assembly; and

FIG. 4 is a flowchart illustrating one embodiment of a method for operating the hand control assembly.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

Embodiments of the present disclosure are described herein in the context of a hand control assembly for an intravascular catheter system. Those of ordinary skill in the art will realize that the following detailed description of the present disclosure is illustrative only and is not intended to be in any way limiting. Other embodiments of the present disclosure will readily suggest themselves to such skilled persons having the benefit of this disclosure. Reference will now be made in detail to implementations of the present disclosure as illustrated in the accompanying drawings.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application-related and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

Although the disclosure provided herein focuses mainly on cryogenics, it is understood that various other forms of energy can be used to ablate diseased heart tissue. These can include radio frequency (RF), ultrasound, pulsed DC electric fields and laser energy, as non-exclusive examples. The present disclosure is intended to be effective with any or all of these and other forms of energy.

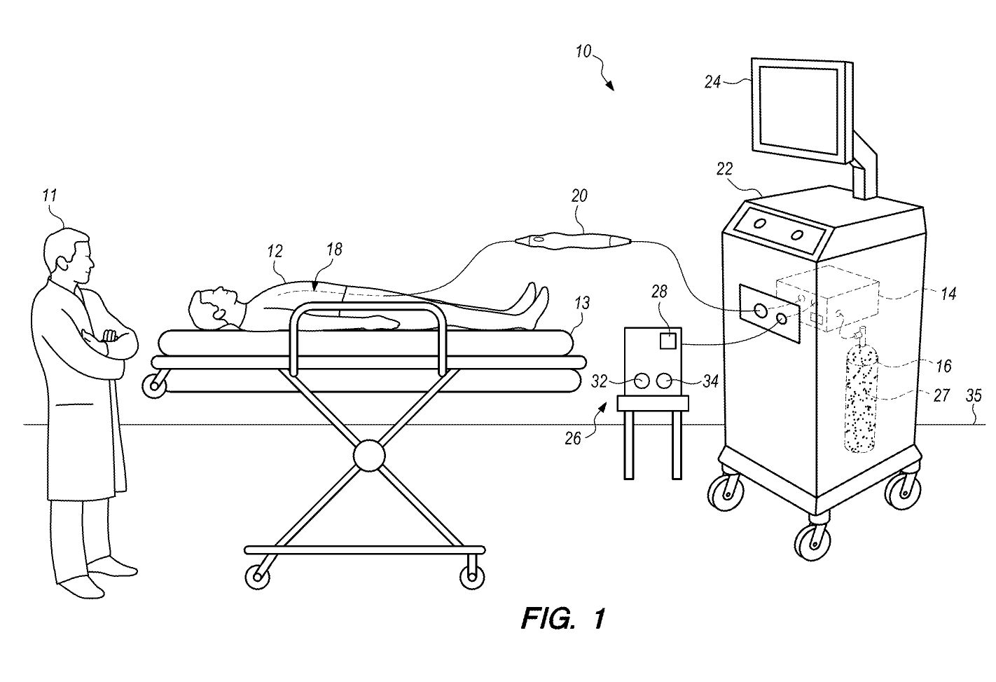

FIG. 1 is a side view illustration of one embodiment of an intravascular catheter system 10 (also sometimes referred to herein as a “catheter system”) for use by an user 11, such as a health care professional, with a patient 12, which can be a human being or an animal. In this embodiment, the user 11 operates and/or controls the catheter system 10 to perform the ablation procedure on the patient 12. While FIG. 1 shows only one user 11, it is understood that a plurality of different users 11 can operate or assist in the operation and/or control of the catheter system 10 at the same or at different times throughout the ablation procedure. In other words, the user 11 illustrated in FIG. 1 can represent any number of different users 11, i.e., a first user, a second user, etc. Further, it is understood that while specific reference is made to the user 11 as a healthcare professional, healthcare professional can include a physician, a physician's assistant, nurse and/or any other suitable person and/or individual.

In the embodiment illustrated in FIG. 1, the patient 12 is positioned on a gurney 13. However, it is understood that the patient 12 can be positioned on any suitable surface, such as a table or a bed, as non-exclusive examples.

Although the catheter system 10 is specifically described herein with respect to the intravascular catheter system, it is understood and appreciated that other types of catheter systems and/or ablation systems can equally benefit by the teachings provided herein. For example, in certain non-exclusive alternative embodiments, the present disclosure can be equally applicable for use with any suitable types of ablation systems and/or any suitable types of catheter systems. Thus, the specific reference herein to use as part of the intravascular catheter system is not intended to be limiting in any manner.

The design of the catheter system 10 can be varied. In certain embodiments, such as the embodiment illustrated in FIG. 1, the catheter system 10 can include one or more of a control system 14, a fluid source 16 (e.g., one or more fluid containers), a balloon catheter 18, a handle assembly 20, a control console 22, a graphical display 24 (also sometimes referred to as a graphical user interface or “GUI”) and a hand control assembly 26. It is understood that although FIG. 1 illustrates the structures of the catheter system 10 in a particular position, sequence and/or order, these structures can be located in any suitably different position, sequence and/or order than that illustrated in FIG. 1. It is also understood that the catheter system 10 can include fewer or additional structures than those specifically illustrated and described herein.

In various embodiments, the control system 14 is configured to monitor and control the various processes of an ablation procedure. More specifically, the control system 14 can monitor and control release and/or retrieval of a cryogenic fluid 27 to and/or from the balloon catheter 18. The control system 14 can also control various structures that are responsible for maintaining or adjusting a flow rate and/or a pressure of the cryogenic fluid 27 that is released to the balloon catheter 18 during the ablation procedure. In various embodiments, the catheter system 10 delivers ablative energy in the form of the cryogenic fluid 27 to cardiac tissue of the patient 12 to create tissue necrosis, rendering the ablated tissue incapable of conducting electrical signals. Additionally, in various embodiments, the control system 14 can control activation and/or deactivation of one or more other processes of the balloon catheter 18. Additionally, or in the alternative, the control system 14 can receive electronic signals, data and/or other information (also sometimes referred to as “sensor output”) from various structures within the catheter system 10. In certain embodiments, the control system 14 and/or the GUI 24 can be electrically connected and/or coupled. In some embodiments, the control system 14 can receive, monitor, assimilate and/or integrate any sensor output and/or any other data or information received from any structure within the catheter system 10 in order to control the operation of the balloon catheter 18. Still further, or in the alternative, the control system 14 can control positioning of portions of the balloon catheter 18 within a circulatory system (also sometimes referred to herein as the “body”) of the patient 12, and/or can control any other suitable functions of the balloon catheter 18.

The fluid source 16 (also sometimes referred to as “fluid container 16”) can include one or more fluid container(s) 16. It is understood that while one fluid container 16 is illustrated in FIG. 1, any suitable number of fluid containers 16 may be used. The fluid container(s) 16 can be of any suitable size, shape and/or design. The fluid container(s) 16 contains the cryogenic fluid 27, which is delivered to the balloon catheter 18 with or without input from the control system 14 during the ablation procedure. Once the ablation procedure has initiated, the cryogenic fluid 27 can be injected or delivered and the resulting gas, after a phase change, can be retrieved from the balloon catheter 18, and can either be vented or otherwise discarded as exhaust. More specifically, the cryogenic fluid 27 delivered to and/or removed from the balloon catheter 18 can include a flow rate that varies. Additionally, the type of cryogenic fluid 27 that is used during the ablation procedure can vary. In one non-exclusive embodiment, the cryogenic fluid 27 can include liquid nitrous oxide. In another non-exclusive embodiment, the cryogenic fluid 27 can include liquid nitrogen. However, any other suitable cryogenic fluid 27 can be used.

The design of the balloon catheter 18 can be varied to suit the design requirements of the catheter system 10. As shown, the balloon catheter 18 is inserted into the body of the patient 12 during the ablation procedure. In one embodiment, the balloon catheter 18 can be positioned within the body of the patient 12 using the control system 14. Stated in another manner, the control system 14 can control positioning of the balloon catheter 18 within the body of the patient 12. Alternatively, the balloon catheter 18 can be manually positioned within the body of the patient 12 by the user 11. In certain embodiments, the balloon catheter 18 is positioned within the body of the patient 12 utilizing at least a portion of the sensor output that is received from the balloon catheter 18. For example, in various embodiments, the sensor output is received by the control system 14, which can then provide the user 11 with information regarding the positioning of the balloon catheter 18. Based at least partially on the sensor output feedback received by the control system 14, the user 11 can adjust the positioning of the balloon catheter 18 within the body of the patient 12 to ensure that the balloon catheter 18 is properly positioned relative to targeted cardiac tissue. While specific reference is made herein to the balloon catheter 18, as noted above, it is understood that any suitable type of medical device and/or catheter may be used.

The handle assembly 20 is handled and used by the user 11 to operate, position and/or control the balloon catheter 18. The design and specific features of the handle assembly 20 can vary to suit the design requirements of the catheter system 10. In the embodiment illustrated in FIG. 1, the handle assembly 20 is separate from, but in electrical and/or fluid communication with the control system 14, the fluid container 16 and the GUI 24. In some embodiments, the handle assembly 20 can integrate and/or include at least a portion of the control system 14 within an interior of the handle assembly 20. In one embodiment, the user 11 can steer and/or navigate the balloon catheter 18 by utilizing the handle assembly 20. It is understood that the handle assembly 20 can include fewer or additional components than those specifically illustrated and described herein.

In the embodiment illustrated in FIG. 1, the control console 22 includes at least a portion of the control system 14, the fluid container 16 and/or the GUI 24. However, in alternative embodiments, the control console 22 can contain additional structures not shown or described herein. Still alternatively, the control console 22 may not include various structures that are illustrated within the control console 22 in FIG. 1. For example, in certain non-exclusive alternative embodiments, the control console 22 does not include the GUI 24.

In various embodiments, the GUI 24 is electrically connected to the control system 14. Additionally, the GUI 24 provides the user 11 of the catheter system 10 with information that can be used before, during and/or after the ablation procedure. For example, the GUI 24 can provide the user 11 with information based on the sensor output, and any other relevant information that can be used before, during and/or after the ablation procedure. The specifics of the GUI 24 can vary depending upon the design requirements of the catheter system 10, or the specific needs, specifications and/or desires of the user 11.

In one embodiment, the GUI 24 can provide static visual data and/or information to the user 11. In addition, or in the alternative, the GUI 24 can provide dynamic visual data and/or information to the user 11, such as video data or any other data that changes over time, e.g., during the ablation procedure. Further, in various embodiments, the GUI 24 can include one or more colors, different sizes, varying brightness, etc., that may act as alerts to the user 11. Additionally, or in the alternative, the GUI 24 can provide audio data or information to the user 11.

As an overview, and as provided in greater detail herein, the hand control assembly 26 allows the user 11 to manually operate and/or control certain stages of the ablation procedure. As used herein, the term “manually” refers to the user 11 using his or her hand or hands to operate and/or control at least a portion and/or a stage of the ablation procedure. Further, “hand” or “hands” refers to any portion of the arm or upper extremities of the user 11, including any attachment thereto and/or extension therefrom, such as a glove or a pointer, as non-exclusive examples. Additionally, as described in greater detail, each ablation procedure can include one or more stages, such as: (i) an inflation stage, (ii) an ablation stage, (iii) a time to isolation, and/or (iv) a thawing stage, as non-exclusive examples. Alternatively, the ablation procedure may also include other stages not specifically mentioned herein.

As utilized herein, the “inflation stage” refers generally to the portion of the ablation procedure, wherein the cryogenic fluid 27 is being delivered from the fluid source 16 to the balloon catheter 18 at a flow rate that does not cause tissue necrosis. More specifically, the cryogenic fluid 27 is being delivered to the inflatable balloon of the balloon catheter 18. During the inflation stage, the user 11 may adjust and/or position the balloon catheter 18 within the body of the patient 12 to achieve positioning of the inflatable balloon adjacent to a targeted tissue of the patient 12. The targeted tissue can include at least a portion of heart tissue of the patient 12 that is to be treated by the catheter system 210, such as an ostium of a pulmonary vein, for example. Once positioned adjacent to the targeted tissue and the pulmonary vein is occluded, ablation of the targeted tissue may be initiated.

The “ablation stage” refers generally to the cryogenic fluid 27 being delivered from the fluid source 16 to the inflatable balloon of the balloon catheter 18 at a flow rate to create tissue necrosis. Tissue necrosis has the effect of rendering targeted tissue incapable of conducting cardiac electrical signals. During ablation of the targeted tissue, the inflatable balloon of the balloon catheter 18 is positioned adjacent to targeted tissue, with the pulmonary vein being occluded.

The “time to isolation” or “time to effect” refers to the moment when cardiac electrical signals during the ablation procedure are lost or “isolated” due to tissue ablation. It is appreciated that the time to isolation is a variable that is determined only through the process of the ablation procedure, and potentially may not actually be achieved in any given ablation procedure. As such, although the ablation procedure can be said to include a time to isolation, it is understood that the specific time to isolation for any given ablation procedure is actually unknown and only a potentiality until it happens (if it does at all) during the ablation procedure. One representative example of time to isolation would be when signals from a left atrium no longer appear in the pulmonary vein due to a circumferential lesion.

Additionally, the “thawing stage” refers generally to the stage of the ablation procedure, wherein targeted tissue of the patient 12 that has been ablated is allowed to thaw to a certain temperature and/or for a certain period of time. The thawing stage can be temperature based, time based, or both. Temperature based means that the ablated heart tissue is allowed to thaw to a certain temperature. Time based means the ablated heart tissue is allowed to thaw for a certain period of time. The temperature and period of time can vary depending on the patient 12 and/or any other ablation parameters. During the thawing stage of the targeted tissue of the patient 12, the cryogenic fluid 27 may be delivered from the fluid source 16 to the inflatable balloon of the balloon catheter 18 and/or retrieved from the inflatable balloon of the balloon catheter 18, but at a flow rate sufficient to maintain the inflatable balloon at least partially or substantially inflated to prevent the balloon catheter 18 from falling out of position and/or to reduce the likelihood of tissue damage to the patient 12.

In certain embodiments, the hand control assembly 26 can be used to initiate and/or terminate any stage of the ablation procedure. As non-exclusive examples, the hand control assembly 26 can be used to initiate and/or terminate the inflation stage, the ablation stage and/or the thawing stage. In other embodiments, the hand control assembly 26 can allow the user 11 to time, measure and/or calculate different events and/or stages of the ablation procedure, such as time to isolation. In yet other embodiments, the hand control assembly 26 can initiate and/or terminate timers and/or other predetermined events. Additionally, and/or alternatively, the hand control assembly 26 can perform any other suitable function of the catheter system 10 that may be manually controlled by the user 11.

The design and specific features of the hand control assembly 26 can vary to suit the design requirements of the catheter system 10. In the embodiment illustrated in FIG. 1, the hand control assembly 26 can include one or more of a controller 28 and a plurality of hand-actuated members, i.e., a first hand-actuated member 32, a second hand-actuated member 34, a third member (not shown), etc. It is recognized that the terms “first hand-actuated member 32,” “second hand-actuated member 34,” “third hand-actuated member,” etc. can be used interchangeably. Each of the plurality of hand-actuated members 32, 34, can be spaced apart from one another. In this embodiment, while specific reference is made herein to the first hand-actuated member 32 and the second hand-actuated member 34, it is further recognized that the hand control assembly 26 can include any number of hand-actuated members, which may allow the user 11 to manually control any suitable function of the catheter system 10. Further, it is understood that the hand control assembly 26 can include fewer or additional components than those specifically illustrated and described herein.

In the embodiment illustrated in FIG. 1, the hand control assembly 26 is designed as a single structure that is coupled and/or connected to the control system 14. The hand control assembly 26 can be electrically and/or mechanically coupled and/or connected to the control system 14 via any suitable manner. Alternatively, the hand control assembly 26 can be coupled and/or connected to other structures of the catheter system 10. Additionally, and/or in the alternative, the hand control assembly 26, can be designed to include various structures, which may be separate from one another.

In the embodiment illustrated in FIG. 1, the user 11, the gurney 13 and the control console 22 are positioned or otherwise situated on or near a support surface 35. The support surface 35 is immovable. Accordingly, the control console 22 can be configured to be positioned on the support surface 35. The hand control assembly 26 can be positioned or otherwise situated away from a support surface 35, such as a floor, for example. The hand control assembly 26 can be positioned away from the support surface 35 via any suitable manner. For example, the hand control assembly 26 can be positioned away from the support surface 35 on any movable or fixed object, including non-exclusive examples such as, a desk, tray, table, or any other suitable object.

Additionally, in this embodiment, the first hand-actuated member 32 and the second hand-actuated member 34 are positioned away from the handle assembly 20 and control console 22. In other words, the first hand-actuated member 32 and the second hand-actuated member 34 are positioned at a location that is away from the handle assembly and/or control console 22. As referred to herein, the term “away from” can refer to being remotely from, spaced from and/or set apart. In alternative embodiments, the first hand-actuated member 32 and the second hand-actuated member 34 can be positioned on and/or integrated with the handle assembly 20. In other alternative embodiments, at least one hand-actuated member 32, 34, can be positioned away from the control console 22, positioned away from the handle assembly 20 and/or positioned on the handle assembly 20. Additionally, and/or alternatively, the first hand-actuated member 32 and the second hand-actuated member 34 can include any combination of positions, such that at least one hand-actuated member 32, 34, is positioned away from the control console 22.

In various embodiments, the controller 28 is configured to receive and/or process electronic or other signals. In certain embodiments, the controller 28 can receive and/or process signals to initiate and/or terminate varying stages of the ablation procedure. More specifically, the controller 28 can receive and/or process signals to initiate and/or terminate the inflation stage, the ablation stage and/or the thawing stage, as non-exclusive examples. In alternative embodiments, the controller 28 can receive and/or process signals to time, measure and/or calculate different stages of the ablation procedure. For example, the controller 28 can calculate and/or measure the time to isolation. Additionally, the controller 28 can receive and/or process other signals to perform any other suitable function.

In various embodiments, the controller 28 can include at least one processor (e.g., microprocessor) that executes software and/or firmware stored in memory of the controller 28. The software/firmware code contains instructions that, when executed by the processor, cause the controller 28 to perform the functions of the control algorithm described herein. The controller 28 may alternatively include one or more application-specific integrated circuits (ASICs), field-programmable gate arrays (FPGAs), digital signal processors (DSPs), hardwired logic, or combinations thereof. The controller 28 may receive information from a plurality of system 10 components and feed the information (e.g., sensor data, signals from the hand control assembly 26, and user inputs from the GUI 24) into a control algorithm which determines at least one control parameter which may in part govern operation of the catheter system 10.

In the embodiment illustrated in FIG. 1, the controller 28 can be integrated and/or included as part of the hand control assembly 26. In other embodiments, the controller 28 can be positioned away from the hand control assembly 26. For example, the controller 28 can be integrated, included as part of and/or positioned within the control system 14, the handle assembly 20 and/or control console 22.

In certain embodiments, the first hand-actuated member 32 can be selectively and/or manually actuated by the user 11 to send a plurality of initiation signals to initiate one or more stages of the ablation procedure. As one non-exclusive example, the first hand-actuated member 32 can be selectively and/or manually actuated by the user 11 to send a first initiation signal to the controller 28. In this embodiment, while specific reference is made herein to sending the first initiation signal, it is recognized that the first hand-actuated member 32 can send one or more initiation signals, i.e., the first initiation signal, a second initiation signal, a third initiation signal, etc. to initiate certain stages of the ablation procedure. It is understood that first initiation signal, the second initiation signal, the third initiation signal, etc., can be used interchangeably. In various embodiments, the controller 28 can process the first initiation signal and can initiate one or more stages of the ablation procedure. Furthermore, the first hand-actuated member 32 can have any suitable design that can enable the user 11 to selectively and/or manually actuate the first hand-actuated member 32.

In various embodiments, the first hand-actuated member 32 can send one or more initiation signals to the controller 28 to initiate certain stages of the ablation procedure depending on how the user 11 actuates the first hand-actuated member 32. More specifically, the function or operation of the first hand-actuated member 32 can depend on how the user 11 actuates the first hand-actuated member 32. In other words, the function or operation of the first hand-actuated member 32 can depend on a first hand-actuated member sequence. As referred to herein, the term “first hand-actuated member sequence” refers to the method or manner in which the first hand-actuated member 32 is actuated, i.e., a number of times, an order, an arrangement, a series, a period of time, etc. and/or any combination thereof. In this embodiment, while specific reference is made to the first hand-actuated member sequence, it is recognized that the hand control assembly 26 can include any number of hand-actuated member sequences, i.e., the first hand-actuated member sequence, a second hand-actuated member sequence, a third hand-actuated member sequence, etc. It is further understood that the first hand-actuated member sequence, the second hand-actuated member sequence, the third hand-actuated member sequence, etc., can be used interchangeably.

In certain embodiments, the first hand-actuated member 32 can initiate varying stages of the ablation procedure depending on the first hand-actuated member sequence the user 11 selects and/or follows to actuate the first hand-actuated member 32. In other words, the first hand-actuated member 32 can initiate varying stages of the ablation procedure depending on the first hand-actuated member sequence selected by the user 11. In some embodiments, the first hand-actuated member sequence and the resulting initiation signal may be predetermined by the user 11 and may depend on certain preferences of user 11 and/or any other ablation parameters. As used herein, “predetermined” can include the user 11 selecting and programming the hand control assembly 26. In other embodiments, the first hand-actuated member sequence and the resulting initiation signal may be preprogrammed. As used herein, “preprogrammed” can mean preset and/or programmed as part of the hand-actuated member assembly 26.

In certain embodiments, the first hand-actuated member sequence the user 11 selects and/or follows to actuate the first hand-actuated member 32 can determine which stage of the ablation procedure will be initiated. As one non-exclusive example, when the first hand-actuated member 32 has been actuated a first time, the first hand-actuated member 32 can send the first initiation signal to the controller 28 to initiate the inflation stage. In the event the first hand-actuated member 32 has been actuated a plurality of times, i.e., a second time, the first hand-actuated member 32 can send the second initiation signal to the controller 28 to initiate the ablation stage. Further, in the event the first hand-actuated member 32 is actuated the plurality of times, i.e., a third time, the first hand-actuated member 32 can send the third initiation signal to the controller 28 to initiate the calculation and/or measurement of the time to isolation.

In another non-exclusive example, the period of time the user 11 depresses and holds down the first hand-actuated member 32 can determine which stage of the ablation procedure will be initiated. For example, if the user 11 depresses and holds down the first hand-actuated member 32 for a second and releases, the inflation stage can be initiated. If the user 11 depresses and holds down the first hand-actuated member 32 for two seconds, the ablation stage can be initiated.

The method and/or manner in which the user 11 actuates the first hand-actuated member 32 can vary. In one embodiment, the first hand-actuated member 32 can include a button wherein certain stages of the ablation procedure can be initiated by the controller 28 depending on the first hand-actuated member sequence the user 11 selects and/or follows to depress the button. In another embodiment, the first hand-actuated member 32 can include a plurality of buttons, with each button corresponding to one of the stages of the ablation procedure, such that alternatingly depressing each of the buttons selectively causes the controller 28 to initiate one of the stages of the ablation procedure. In still another embodiment, the first hand-actuated member 32 can include a switch that can be selectively and/or manually moved or slid to enable the user 11 to cause the controller 28 to initiate one of the stages of the ablation procedure. Alternatively, the first hand-actuated member 32 can have any other suitable design that enables the user 11 to selectively and/or manually actuate the first hand-actuated member 32 to cause the controller 28 to initiate varying stages of the ablation procedure.

In various embodiments, the second hand-actuated member 34 can also be selectively and/or manually actuated by the user 11 to send a plurality of termination signals to terminate one or more stages of the ablation procedure. As one non-exclusive example, the second hand-actuated member 34 can be selectively and/or manually actuated by the user 11 to send a first termination signal to the controller 28. In this embodiment, while specific reference is made herein to sending the first termination signal, it is recognized that the second hand-actuated member 34 can send one or more termination signals, i.e., the first termination signal, a second termination signal, a third termination signal, etc. to terminate certain stages of the ablation procedure, which can be collectively referred to herein as a “termination signal.” It is further understood, that the first termination signal, the second termination signal, the third termination signal, etc., can be used interchangeably. Once actuated, the second hand-actuated member 34 can send the first termination signal to the controller 28. In various embodiments, the controller 28 can then process the first termination signal to terminate certain stages of the ablation procedure. Additionally, the second hand-actuated member 34 can have any suitable design so as to enable the user 11 to selectively and/or manually actuate the second hand-actuated member 34.

In certain embodiments, the second hand-actuated member 34 can terminate certain stages of the ablation procedure depending on the second hand-actuated member sequence the user 11 selects and/or follows to actuate the second hand-actuated member 34. More specifically, the function or operation of the second hand-actuated member 34 can depend on how the user 11 actuates the second hand-actuated member 34. In other words, the second hand-actuated member 34 can terminate certain stages of the ablation procedure depending on the second hand-actuated member sequence selected by the user 11 and/or preprogrammed as part of the hand-actuated member assembly 26. As referred to herein, the term “second hand-actuated member sequence” can include the method or manner in which the second hand-actuated member 34 is actuated, i.e., a number of times, an order, an arrangement, a series, a length of time, etc. and/or any combination thereof.

More specifically, in one non-exclusive example, in the event the second hand-actuated member 34 has been actuated a first time during the inflation stage, the second hand-actuated member 34 can send the first termination signal to the controller 28 to terminate the inflation stage. Alternatively, when the second hand-actuated member 34 has been actuated a first time during the ablation stage, the second hand-actuated member 34 can send the second termination signal to the controller 28 to terminate and/or stop the ablation stage. In some embodiments, when the second hand-actuated member 34 has been actuated a first time during the ablation stage, the thawing stage may also be initiated. The thawing stage can be initiated at any time at or after the ablation stage has been terminated or stopped, i.e., substantially simultaneously with the termination of the ablation stage, for example. In the event the second hand-actuated member 34 is actuated a plurality of times, i.e., a second time after the ablation stage has initiated and/or a first time during the thawing stage, the second hand-actuated member 34 can send a third termination signal to the controller 28 to terminate the ablation stage and/or the thawing stage. In various embodiments, should the inflation stage, the ablation stage and/or the thawing stage be terminated or stopped, the catheter system 10 may return to an idle position, at which time the controller 28 can reset the hand control assembly 26. In other words, the first hand-actuated member sequence of the first hand-actuated member 32 and/or the second hand-actuated member sequence of the second hand-actuated member 34 selected and/or followed by the user 11 is reset or recalibrated.

The method and/or manner in which the user 11 actuates the second hand-actuated member 34 can vary. In certain embodiments, the second hand-actuated member 34 can include a button, wherein certain stages of the ablation procedure can be terminated by the controller 28 depending on the second hand-actuated member sequence selected and/or followed by the user 11 to depress the button. In other embodiments, the second hand-actuated member 34 can include a plurality of buttons, with each button corresponding to one of the stages of the ablation procedure, such that alternatingly depressing each of the buttons selectively causes the controller 28 to terminate one of the stages of the ablation procedure. In still other embodiments, the second hand-actuated member 34 can include a switch that can be selectively and/or manually moved or slid to enable the user 11 to cause the controller 28 to terminate one of the stages of the ablation procedure. Alternatively, the second hand-actuated member 34 can have any other suitable design that enables the user 11 to selectively and/or manually actuate the second hand-actuated member 34 to cause the controller 28 to terminate certain stages of the ablation procedure.

In one non-exclusive embodiment, the hand control assembly 26 may include only the first hand-actuated member 32. In this embodiment, the first hand-actuated member 32 can initiate and terminate certain stages of the ablation procedure depending on the first hand-actuated member sequence the user 11 selects and/or follows to actuate the first hand-actuated member 32. In other words, the first hand-actuated member 32 can initiate and terminate certain stages of the ablation procedure depending on the first hand-actuated member sequence selected by the user 11 and/or preprogrammed as part of the hand-actuated member assembly 26. For instance, when the first hand-actuated member 32 has been actuated a first time, the first hand-actuated member 32 can send the first initiation signal to the controller 28 to initiate the inflation stage. In the event the first hand-actuated member 32 has been actuated a plurality of times, the first hand-actuated member 32 can send at least one of: the second initiation signal to the controller 28 to initiate the ablation stage and/or the third initiation signal to the controller 28 to initiate the calculation and/or measurement of the time to isolation. Additionally, in the event the first hand-actuated member 32 has been actuated the plurality of times, the first hand-actuated member can send at least one of: the first termination signal to the controller 28 to terminate the inflation stage, the second termination signal to the controller 28 to terminate the ablation stage and/or the third termination signal to the controller 28 to terminate the thawing stage.

In another non-exclusive embodiment, the first hand-actuated member 32 and/or the second hand-actuated member 34 can allow the user 11 to control a flow rate of the cryogenic fluid 27 to and/or from the balloon catheter 18. In other words, the first hand-actuated member 32 and/or the second hand-actuated member 34 can control the cryogenic fluid 27 that is released to the balloon catheter 18 during the ablation procedure, which may adjust (i.e., increase or decrease) and/or maintain an inflatable balloon size, a temperature and/or a pressure within the inflatable balloon of the balloon catheter 18. As used herein, the term “control” can include to initiate, increase and/or decrease. More specifically, the user 11 can depress and hold down the first hand-actuated member 32 and/or second hand-actuated member 34 in order to achieve or reach a desired flow rate, temperature and/or pressure. Further, the user 11 can depress and hold down the first hand-actuated member 32 and/or second hand-actuated member 34 in order to achieve or reach the desired inflatable balloon size. While in this embodiment, the method of depressing is described, it is understood that the first hand-actuated member 32 and/or second hand-actuated member 34 may be moved, slid, etc. and held. Once the desired flow rate, inflatable balloon size, temperature and/or pressure is achieved, the user 11 can release the first hand-actuated member 32 and/or the second hand-actuated member 34. As the first hand-actuated member 32 and/or the second hand-actuated member 34 is released, the desired flow rate, inflatable balloon size, temperature and/or pressure may be maintained. As used herein, the term “maintain” means to keep, sustain, preserve, etc., substantially the same flow rate, inflatable balloon size, temperature and/or pressure as at the time the first hand-actuated member 32 and/or the second hand-actuated member 34 was released.

In one embodiment, the first hand-actuated member 32 can be depressed and held down a first time to send a first depression signal to the controller 28 to control, i.e., initiate and/or increase, the flow of cryogenic fluid 27 until the desired flow rate, inflatable balloon size, temperature and/or pressure for the initiation stage is achieved or reached. Once the desired flow rate, inflatable balloon size, temperature and/or pressure for the initiation stage is achieved, the user 11 can release the first hand-actuated member 32. As the first hand-actuated member 32 is released, the first hand-actuated member 32 can send a first release signal to the controller 28 to maintain the desired flow rate, inflatable balloon size, temperature and/or pressure for the inflation stage. Further, the first hand-actuated member 32 can be depressed and held down a second time to send the first depression signal to the controller 28 to control, i.e., increase, the flow rate of the cryogenic fluid 27 until the desired flow rate, inflatable balloon size, temperature and/or pressure for the ablation stage is achieved. Once the desired flow rate, inflatable balloon size, temperature and/or pressure for the ablation stage is achieved, the user 11 can release the first hand-actuated member 32. As the first hand-actuated member 32 is released, the first hand-actuated member 32 can send the first release signal to the controller 28 to maintain the desired flow rate, inflatable balloon size, temperature and/or pressure for the ablation stage.

Still further, the second hand-actuated member 34 can be depressed and held down at any point during the ablation procedure to send a second depression signal to the controller 28 to control, i.e., decrease, the flow rate of the cryogenic fluid 27. For example, the second hand-actuated member 34 can be depressed and held down by the user 11 until the desired flow rate, inflatable balloon size, temperature and/or pressure for thawing stage has been achieved. Once the desired flow rate, inflatable balloon size, temperature and/or pressure for the thawing stage is achieved, the user 11 can release the second hand-actuated member 34 to send a second release signal to the controller 28 to maintain the desired flow rate, inflatable balloon size, temperature and/or pressure for the thawing stage.

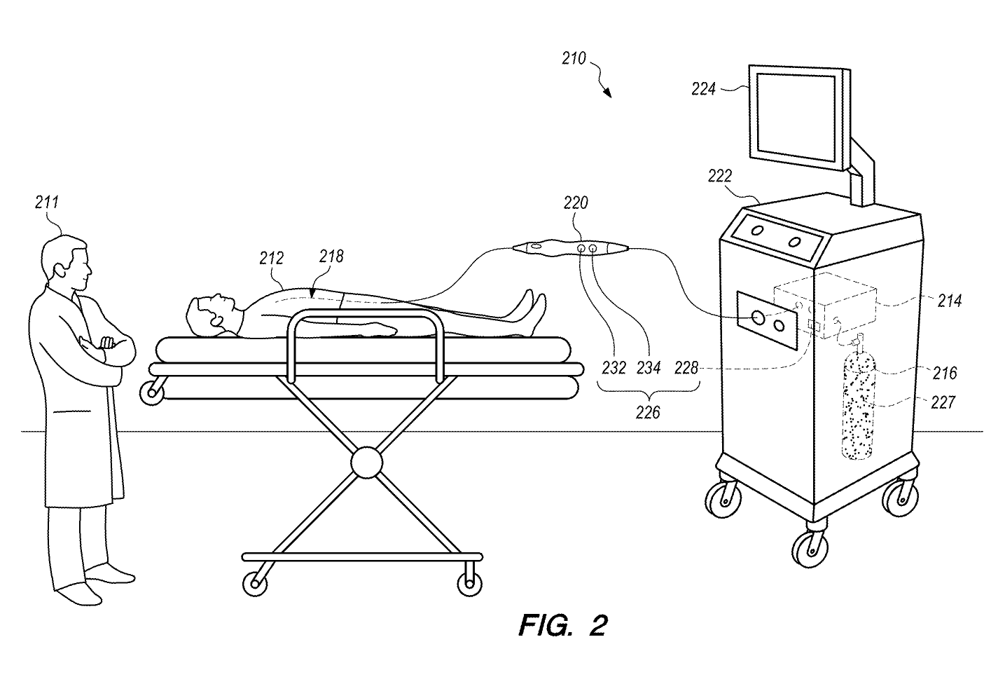

FIG. 2 is a schematic side view of the user 211, the patient 212 and another embodiment of the catheter system 210. In the embodiment illustrated in FIG. 2, the catheter system 210 includes the control system 214, the fluid source 216, the balloon catheter 218, the handle assembly 220, the control console 222, the GUI 224 and the hand control assembly 226. In FIG. 2, the hand control assembly 226 is positioned away from the control console 222 or at a location that is away from the control console 222. However, in the embodiment illustrated in FIG. 2, the hand control assembly 226 is integrated or included with the handle assembly 220. In other words, the first hand-actuated member 232 and the second hand-actuated member 234 are positioned on the handle assembly 220.

Additionally, in the embodiment illustrated in FIG. 2, the controller 228 is integrated and/or included as part of the control system 214. In other embodiments, the controller 228 can be separate and/or apart from the control system 214, and integrated and/or included as part of the handle assembly 220, for example. Additionally, and/or alternatively, the controller 228 can be integrated and/or included as part of any other suitable structure in the catheter system 210.

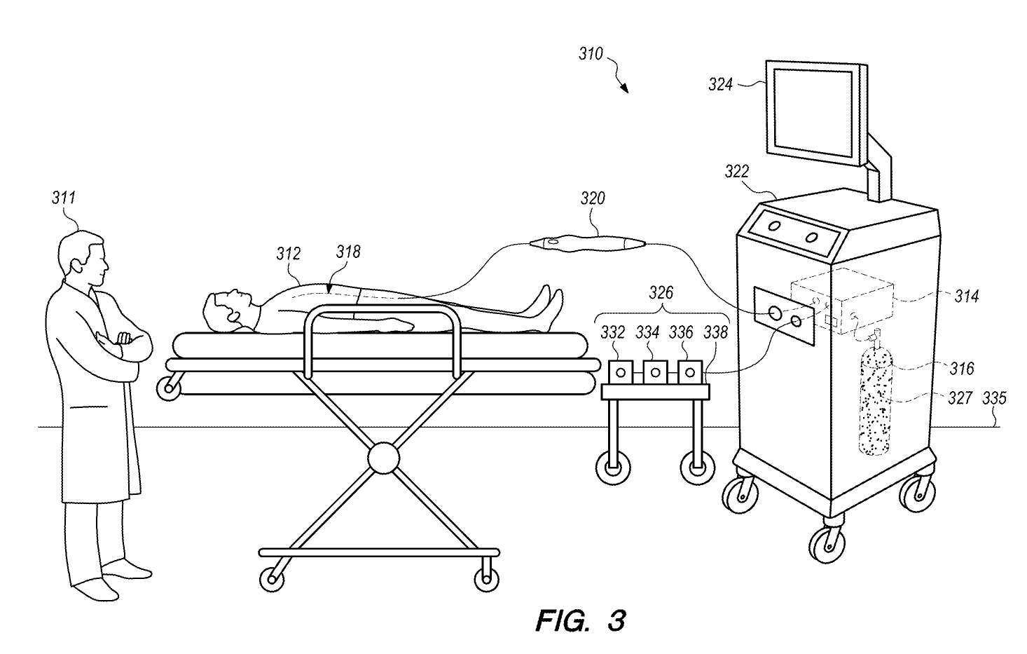

FIG. 3 is a schematic side view of the user 311, the patient 312 and another embodiment of the catheter system 310. In the embodiment illustrated in FIG. 3, the catheter system 310 includes the control system 314, the fluid source 316, the balloon catheter 318, the handle assembly 320, the control console 322, the GUI 324 and the hand control assembly 326. However, in the embodiment illustrated in FIG. 3, the hand control assembly 326 includes the controller 328 and the plurality of hand-actuated members, i.e., the first hand-actuated member 332, the second hand-actuated member 334 and a third hand-actuated member 336, and a member support surface 338.

In this embodiment, the hand control assembly 326 includes several structures which are coupled and/or connected to each other and the controller 328. Alternatively, the first hand-actuated member 332, the second hand-actuated member 334 and the third hand-actuated member 336 can be separately coupled and/or connected to the controller 328. The controller 328, the first hand-actuated member 332, the second hand-actuated member 334 and the third hand-actuated member 336 can be coupled and/or connected via any suitable manner.

In the embodiment illustrated in FIG. 3, the controller 328 can be integrated and/or included as part of the control system 314. In other embodiments, the controller 328 can be separate and/or apart from the control system 314, and integrated and/or included as part of the control console 322, for example. Additionally, and/or alternatively, the controller 328 can be integrated and/or included as part of any other suitable structure in the catheter system 310.

Additionally, in this embodiment, the hand control assembly 326 is positioned away from the handle assembly 320, control console 322 and the support surface 335. Stated another way, the hand control assembly 326 is positioned at a location that is away from the handle assembly 320, the control console 322 and/or the support surface 335.

In certain embodiments, the third hand-actuated member 336 can be selectively and/or manually actuated by the user 311 to send a plurality of timer signals. As one non-exclusive example, the third hand-actuated member 336 can be selectively and/or manually actuated by the user 311 to send a first timer signal to the controller 328. In this embodiment, while specific reference is made herein to sending the first timer signal, it is recognized that the third hand-actuated member 336 can send one or more timer signals, i.e., the first timer signal, a second timer signal, etc. to initiate and/or terminate timers. It is further understood that the first timer signal, the second timer signal, etc., can be used interchangeably.

Once actuated, the third hand-actuated member 336 can send the plurality of timer signals to the controller 328. In various embodiments, the controller 328 can then process the plurality of timer signals to initiate and/or terminate certain timers. As used herein, “timers” can include the monitoring and/or recording of time for any suitable function of the catheter system 310. In one embodiment, the timer can be configured to monitor elapsed time during the ablation procedure until the time to isolation is achieved. In another embodiment, the timer can be configured to monitor elapsed time from the beginning of the ablation procedure to when targeted tissue is effectively isolated and non-conducting, i.e., at the time to isolation. In various embodiments, the third hand-actuated member 336 can be substantially similar in design and/or configuration to the first hand-actuated member 332 and the second hand-actuated member 334. Alternatively, the third hand-actuated member 336 can have any other suitable design so as to enable the user 311 to selectively and/or manually actuate the third hand-actuated member 336.

In some non-exclusive embodiments, the third hand-actuated member 336 can be configured to specifically provide the user 311 with the means to selectively and/or manually actuate the third hand-actuated member 336 to cause the controller 328 to initiate and/or terminate timers during varying stages of the ablation procedure. In certain embodiments, the third hand-actuated member 336 can initiate and/or terminate timers during varying stages of the ablation procedure depending on the third hand-actuated member sequence the user 311 selects and/or follows to actuate the third hand-actuated member 336. In other words, the third hand-actuated member 336 can initiate and/or terminate timers during varying stages of the ablation procedure depending on the third hand-actuated member sequence selected and/or followed by the user 311. For example, in certain embodiments, the third hand-actuated member 336 can initiate or terminate certain timers depending on the number of times the user 311 actuates, i.e., depresses, the third hand-actuated member 336. Alternatively, the third hand-actuated member 336 can initiate or terminate timers depending on the period of time the user 311 holds down the third hand-actuated member 336.

The method and/or manner in which the user 311 actuates the third hand-actuated member 336 can vary. In certain embodiments, the third hand-actuated member 336 can include a button or a switch wherein timers during varying stages of the ablation procedure can be initiated or terminated by the controller 328 depending on the third hand-actuated member sequence selected and/or followed by the user 311 to depress, move or slide the button or switch. More specifically, in one non-exclusive embodiment, when the third hand-actuated member 336 has been actuated a first time, the third hand-actuated member 336 can send the first timer signal to the controller 328 to initiate the timer. In the event the third hand-actuated member 336 has been actuated a plurality of times, i.e., second time, the third hand-actuated member 336 can send the second timer signal to the controller 328 to terminate the timer. Additionally, and/or alternatively, the third hand-actuated member 336 can have any other suitable design that effectively enables the user 311 to selectively and/or manually actuate the third hand-actuated member 336 to cause the controller 328 to initiate or terminate timers.

In one non-exclusive embodiment, the third hand-actuated member 336 can function to activate and/or deactivate the hand control assembly 326. More specifically, while the hand control assembly 326 is in the idle position, the user 311 can actuate the third hand-actuated member 336 to send a deactivation signal to the controller 328 to deactivate the hand control assembly 326. Additionally, the user 311 can actuate the third hand-actuated member 336 to send an activation signal to the controller 328 to activate or reactivate the hand control assembly 326. For example, the user 311 can depress the third hand-actuated member 336 a first time for a certain period of time, i.e., “x” amount of seconds, to send the deactivation signal to the controller 328 to deactivate the hand control assembly 326. This may have the effect of relatively minimizing any accidental initiation of the inflation stage and/or ablation stage by the user 311. The user 311 can also depress the third hand-actuated member 336 a plurality of times, i.e., second time, to send the activation signal to the controller 328 to activate or reactivate the hand control assembly 326.

In the embodiment illustrated in FIG. 3, the hand control assembly 326 also includes the member support surface 338. The member support surface 338 can include any suitable movable or fixed object, which may include a desk, a tray, a table, a stool, a bed, a drawer, a gurney, a cart, a stand, or a cabinet, as non-exclusive examples. In various embodiments, the hand control assembly 326 can be supported by the member support surface 338. In other words, the hand control assembly may positioned or otherwise situated on the member support surface 338. As referred to herein, the hand control assembly 326 being configured to be positioned away from the support surface 335 can include being positioned or otherwise situated on the member support surface 338.

In FIG. 3, the member support surface 338 is movable relative to the support surface 335. In alternative embodiments, the member support surface 338 may be in a substantially fixed position, which may not be movable relative to the support surface 335. In some embodiments, such as the embodiment illustrated in FIG. 3, the member support surface 338 can be positioned on the support surface 335. In other embodiments, the member support surface 338 can be positioned on any other suitable movable or fixed object. For example, the member support surface 338 may include a tray, which may then be positioned on a cart, bed, table, etc. Accordingly, in such embodiments, the member support surface 338 may be movable relative to other structures of the catheter system 310.

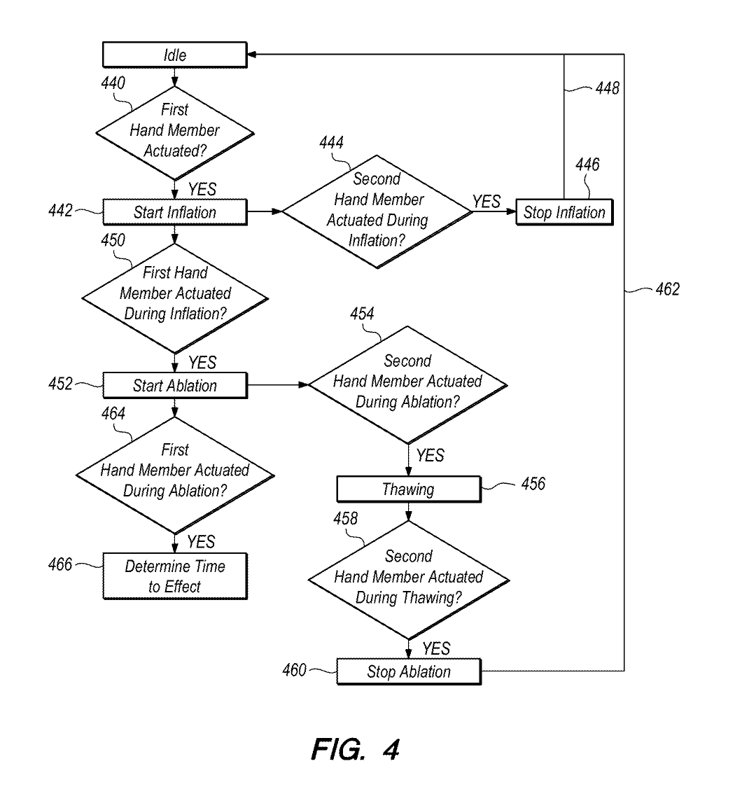

FIG. 4 is a flowchart illustrating one embodiment of a method for operating the hand control assembly 426. It is appreciated that the order of the steps illustrated and described in FIG. 4 is not necessarily indicative of how the hand control assembly 426 operates chronologically, as one or more of the steps can be combined, reordered, repeated and/or performed simultaneously without deviating from the intended breadth and scope of the hand control assembly 426 and method. It further is recognized that the flowchart shown in FIG. 4 is merely one representative example of how the hand control assembly 426 can be utilized within the catheter system 410 and is not intended to be limiting in any manner.

At step 440, a determination is made whether the first hand-actuated member is actuated. The first hand-actuated member is actuated when the first hand-actuated member is depressed, moved, slid, etc. by the user.

At step 442, in the event the first hand-actuated member has been actuated the first time, the first hand-actuated member sends the first initiation signal to the controller to initiate the inflation stage, i.e., “Start Inflation”.

At step 444, a determination is made whether the second hand-actuated member is actuated. The second hand-actuated member is actuated when the second hand-actuated member is depressed, moved, slid, etc. by the user during the inflation stage or the first time.

At step 446, in the event the second hand-actuated member is actuated during the inflation stage or the first time, the second hand-actuated member sends the first termination signal to the controller to terminate or stop the inflation stage, i.e., to “Stop Inflation”.

At step 448, in the event the inflation stage is terminated or stopped, the catheter system may return to the idle position, i.e., “Idle”, at which time the controller can reset or recalibrate the hand control assembly.

At step 450, a determination is made whether the first hand-actuated member has been actuated during the inflation stage or the second time.

At step 452, in the event the first hand-actuated member is actuated during the inflation stage or the second time, the first hand-actuated member sends the second initiation signal to the controller to initiate the ablation stage, i.e., to “Start Ablation”.

At step 454, a determination is made whether the second hand-actuated member has been actuated during the ablation stage or the first time after the ablation stage has been initiated.

At step 456, in the event the second hand-actuated member is actuated during the ablation stage or the first time after the ablation stage has initiated, the second hand-actuated member sends the second termination signal to the controller to terminate the ablation stage. In some embodiments, the second termination signal may also initiate the thawing stage. The second termination signal can initiate the thawing stage substantially at or after the time the ablation stage has been terminated or stopped.

At step 458, a determination is made whether the second hand-actuated member has been actuated the first time during the thawing stage or the second time after the ablation stage has initiated.

At step 460, in the event the second hand-actuated member is actuated during the thawing stage and/or the second time after the ablation stage has initiated, the second hand-actuated member sends the third termination signal to the controller to terminate or stop the ablation stage and/or the thawing stage, i.e., to “Stop Ablation”.

At step 462, in the event the ablation stage and/or the thawing stage are terminated or stopped, the catheter system may return to the idle position, i.e., “Idle”, at which time the controller can reset or recalibrate the hand control assembly.

At step 464, a determination is made whether the first hand-actuated member has been actuated during the ablation stage or the third time.

At step 466, in event the first hand-actuated member is actuated during the ablation stage or the third time, the first hand-actuated member sends the third initiation signal to the controller to initiate the calculation and/or measurement of the time to isolation or time to effect.

It is understood that although a number of different embodiments of the catheter system and/or the hand control assembly have been illustrated and described herein, one or more features of any one embodiment can be combined with one or more features of one or more of the other embodiments, provided that such combination satisfies the intent of the present disclosure.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

I claim:

1. An ablation system for use in performing an ablation procedure, the ablation system comprising:
an ablation catheter having a handle assembly, and an ablation element configured to be advanced within a body of a patient and to deliver ablative energy to target tissue of the patient;
a control console operatively coupled to the ablation catheter and including an ablation energy supply; and
a controller operatively coupled to the control console and configured to control one or more stages of the ablation procedure; and
first and second hand-actuated members configured to be positioned remotely from the control console at a location where the first and second hand-actuated members can be manually operated by a hand of a user while the user is manipulating the ablation catheter within the patient, each of the first and second hand-actuated members being operatively coupled to the controller and configured to be selectively actuated by the user to selectively send one or both of: (i) at least one initiation signal to the controller to initiate a stage of the ablation procedure, and (ii) at least one termination signal to the controller to terminate a stage of the ablation procedure.

2. The ablation system of claim 1, wherein the first and second hand-actuated members are disposed in one of: (i) the handle assembly; and (ii) a hand control assembly that is configured to be located remotely from the control console.

3. The ablation system of claim 2, wherein one of the first and second hand-actuated members is configured to be selectively actuated by the user to send the at least one initiation signal to the controller, and the other of the first and second hand-actuated members is configured to be selectively actuated by the user to send the at least one termination signal to the controller.

4. The ablation system of claim 2, wherein the ablation procedure includes an inflation stage, and wherein the at least one initiation signal initiates the inflation stage, and wherein the at least one termination signal terminates the inflation stage.

5. The ablation system of claim 2, wherein the ablation procedure includes an ablation stage, and wherein the at least one initiation signal initiates the ablation stage, and wherein the at least one termination signal terminates the ablation stage.

6. The ablation system of claim 2, wherein the ablation procedure includes a time to isolation, and wherein the at least one initiation signal initiates a calculation of the time to isolation.

7. The ablation system of claim 2, wherein the ablation procedure includes an ablation stage and a thawing stage, and wherein the at least one termination signal terminates the ablation stage and substantially simultaneously initiates the thawing stage.

8. The ablation system of claim 2, wherein the at least one initiation signal includes a timer signal to the controller to initiate a timer configured to monitor time elapsed during a stage of the ablation procedure.

9. The ablation system of claim 2, wherein one of the first and second hand-actuated members is configured to be selectively actuated by the user to send at least one of (i) a plurality of initiation signals to the controller to initiate at least one stage of the ablation procedure, or (ii) a plurality of termination signals to the controller to terminate at least one stage of the ablation procedure.

10. The ablation system of claim 1, wherein the controller and the first and second hand-actuated members are incorporated into a hand control assembly that is configured to be located remotely from the control console.

11. An ablation system for use in performing an ablation procedure, the ablation system comprising:
an ablation catheter having a handle assembly, and an ablation element configured to be advanced within a body of a patient and to deliver ablative energy to target tissue of the patient;
a control console operatively coupled to the ablation catheter and including an ablation energy supply; and
a hand control assembly operatively coupled to the control console and configured to be positioned remotely from the handle assembly and the control console at a location where the hand control assembly can be manually operated by a hand of a user while the user is manipulating the ablation catheter within the patient, the hand control assembly comprising:
a controller configured to control one or more stages of the ablation procedure; and
a plurality of hand-actuated members that are each configured to be selectively actuated by the user to send one or both of (i) an initiation signal to the controller to initiate at least one stage of the ablation procedure, and (ii) a termination signal to the controller to terminate at least one stage of the ablation procedure.

12. The ablation system of claim 11, wherein the ablation procedure includes a plurality of stages including an inflation stage, an ablation stage and a thawing stage, and wherein one of the plurality of hand-actuated members includes a first hand-actuated member configured to be selectively actuated by the user to send a first initiation signal to the controller to initiate one of the plurality of stages, and to be selectively actuated by the user to send a second initiation signal to the controller to initiate a different one of the plurality of stages.

13. The ablation system of claim 12, wherein the ablation procedure includes a time to isolation, and wherein the first hand-actuated member is configured to be selectively actuated by the user to send a third initiation signal to the controller to initiate a calculation of the time to isolation.

14. The ablation system of claim 12, wherein the plurality of hand-actuated members further includes a second hand-actuated member configured to be selectively actuated by the user to send a first termination signal to the controller to terminate one of the plurality of stages, and to be selectively actuated by the user to send a second termination signal to the controller to terminate a different one of the plurality of stages.

15. The ablation system of claim 11, wherein the ablation procedure includes a plurality of stages including an inflation stage, an ablation stage and a thawing stage, and wherein the plurality of hand-actuated members includes a first hand-actuated member and a second hand-actuated member, wherein the first hand-actuated member is configured to be selectively actuated by the user to send a first initiation signal to the controller to initiate one of the plurality of stages, and the second hand-actuated member is configured to be selectively actuated by the user to send a first termination signal to the controller to terminate the one of the plurality of stages.

16. A method for controlling at least one stage of an ablation procedure, the method comprising:
manipulating an ablation catheter so as to position an ablation element thereof at a location proximate target tissue of a patient; and
selectively actuating a first hand-actuated member to send at least one at least one of (i) an initiation signal to a controller to initiate at least one stage of the ablation procedure, or (ii) a termination signal to the controller to terminate at least one stage of the ablation procedure,
wherein the first hand-actuated member is operatively coupled to the controller and is positioned at a location where the first hand-actuated member can be actuated by a hand of a user while the user is manipulating the ablation catheter within the patient.

17. The method of claim 16, wherein the ablation procedure includes a plurality of stages including an inflation stage, an ablation stage and a thawing stage, and wherein selectively actuating the first hand-actuated member includes selectively actuating the first hand-actuated member to send a first initiation signal to the controller to initiate one of the plurality of stages, and selectively actuating the first hand-actuated member to send a second initiation signal to the controller to initiate a different one of the plurality of stages.

18. The method of claim 17, wherein selectively actuating the first hand-actuated member includes selectively actuating the first hand-actuated member to send a third initiation signal to the controller to calculate a time to isolation.

19. The method of claim 17, wherein selectively actuating the first hand-actuated member includes selectively actuating the first hand-actuated member to send a first termination signal to the controller to terminate the one of the plurality of stages, and selectively actuating the first hand-actuated member to send a second termination signal to the controller to terminate the different one of the plurality of stages.

20. The method of claim 16, wherein the ablation procedure includes a plurality of stages including an inflation stage, an ablation stage and a thawing stage, and wherein selectively actuating the first hand-actuated member includes selectively actuating the first hand-actuated member to send an initiation signal to the controller to initiate one of the plurality of stages, and wherein the method further comprises selectively actuating a second hand-actuated member to send a termination signal to the controller to terminate the one of the plurality of stages, wherein the second hand-actuated member is operatively coupled to the controller and is positioned at a location where the second hand-actuated member can be actuated by a hand of a user while the user is manipulating the ablation catheter within the patient.
